SEQUENCE LISTING (4 sequences)

SEQ ID NO: 1 - DNA, 157 bp, Dengue Virus
  misc_feature (1)..(157)
ggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctc

SEQ ID NO: 2 - DNA, 18 bp, Artificial Sequence
  misc_feature (1)..(18): Synthetic Oligonucleotide
ggttagaggagacccctc

SEQ ID NO: 3 - DNA, 18 bp, Artificial Sequence
  misc_feature (1)..(18): Synthetic Oligonucleotide
gagacagcaggatctctg

SEQ ID NO: 4 - DNA, 19 bp, Artificial Sequence
  misc_feature (1)..(18): Synthetic Oligonucleotide
tcccagcgtcaatatgctg

(19) United States
(12) Patent Application Publication
(10) Pub. No.: US 2009/0197245 A1
(43) Pub. Date: Aug. 6, 2009

(54) Rapid detection of dengue virus

(75) Inventor: James C. Mcavin, San Antonio, TX (US)

Correspondence Address: DEPARTMENT OF THE AIR FORCE, AFMC LO/JAZ, BLdg 11, Room D18, WRIGHT-PATTERSON AFB, OH, 45433-7109, US

(73) Assignee: United States, as represented by the Secretary of the Air Force

(21) Appl. No.: 11/998,985

(22) Filed: Nov. 21, 2007

Related U.S. Application Data
(60) Provisional application No. 60/877,017, filed on Nov. 28, 2006.

(51) Int. Cl. C12Q 1/70

(52) U.S. Cl. 435/5

(57) ABSTRACT

One example of a solution provided here comprises providing a single-stranded oligonucleotide, the oligonucleotide being complementary to a portion of SEQ ID NO:1, and contacting the oligonucleotide with a nucleic acid comprising the sequence of SEQ ID NO:1, under conditions that permit hybridization of the oligonucleotide with the nucleic acid. Another example comprises providing a single-stranded oligonucleotide comprising the sequence of SEQ ID NO:4, and contacting the oligonucleotide with a nucleic acid comprising the sequence of SEQ ID NO:1, under conditions that permit hybridization of the oligonucleotide with the nucleic acid.

### RELATED APPLICATION, AND RIGHTS OF THE GOVERNMENT

This application claims the benefit under 35 U.S.C. §119(e) of provisional Patent Application Ser. No. 60/877,017, filed Nov. 28, 2006, the entire text of which is incorporated herein by reference. The invention described herein may be manufactured and used by or for the Government of the United States for all governmental purposes without the payment of any royalty.

### BACKGROUND OF THE INVENTION

The invention relates to assays and more particularly to screening biological samples.

An approved dengue fever vaccine or prophylactic drug does not currently exist therefore the only effective protection is avoidance of dengue virus through surveillance of infected mosquitoes and efficacious patient management requires rapid, sensitive, and specific diagnostics. Dengue fever (DF) and the more severe forms of the disease, dengue hemorrhagic fever (DHF) and dengue shock syndrome (DSS) occur in all tropical and subtropical regions through infection by one or more of four viral serotypes, dengue serotypes 1-4 (Gubler, Clin Microbiol Rev 1998 July: 480-496). Dengue virus is transmitted in a cycle that primarily involves humans and mosquito vectors, most significantly Aedes aegypti in developing urban and semi-urban areas. The prevalence of dengue virus is now comparable to malaria making DF the most significant mosquito-borne viral disease, threatening two-fifths of the world's human population (www.cdc.gov/ncidod/dvbid/dengue/slideset/index.html, www.who.int/inf-fs/en/fact117.html). It is estimated that 50-100 million people are affected annually with DF and 300,000 with DHF/DSS. Dengue virus surveillance and DF/DHF diagnoses are problematic. Symptoms are usually nonspecific and serologic analyses or virus isolation from mosquitoes can take a week or more (Gibbons et al BMJ 2002; 324:1563-6). Antibody cross-reaction occurs across genotypic and symptomatic near neighbors creating ambiguity in immunoassay-based analyses. Reverse transcriptase-polymerase chain reaction (RT-PCR) assays have been developed for use on laboratory-based instrumentation.

However, there is a need for simpler Dengue assay technology, especially in areas where clinical laboratory facilities are not available, for example.

### SUMMARY OF THE INVENTION

One example of a solution provided here comprises providing a single-stranded oligonucleotide, the oligonucleotide being complementary to a portion of SEQ ID NO:1, and contacting the oligonucleotide with a nucleic acid comprising the sequence of SEQ ID NO:1, under conditions that permit hybridization of the oligonucleotide with the nucleic acid. Another example comprises providing a single-stranded oligonucleotide comprising the sequence of SEQ ID NO:4, and contacting the oligonucleotide with a nucleic acid comprising the sequence of SEQ ID NO:1, under conditions that permit hybridization of the oligonucleotide with the nucleic acid.

### BRIEF DESCRIPTION OF THE DRAWINGS

FIG. 1 is a diagram showing examples of a target sequence (in bold type), RT-PCR forward primer (sense), probe (anti-sense), and reverse primer (anti-sense) sequences (in bold caps).

### DETAILED DESCRIPTION

There is a need for simpler Dengue assay technology. There is a need for field-deployable surveillance to achieve timely assessments of transmission risk, time-critical implementation of focused mosquito control measures and clinical response in a potential outbreak situation. An example of a solution provided here is a field-deployable, field-sustainable dengue virus assay for rapid, sensitive and specific screening in mosquito vectors and human sera on field-deployable instrumentation. Another example of a solution provided here comprises assay primers specifically amplifying a region of the dengue genome, excluding genotypically similar and clinically significant species, and hybridization of a probe to the amplification product, detecting the presence of the target. Another example of a solution provided here comprises an assay that detects all dengue virus serotypes, is adaptable for use on a field-durable, real-time analytic platform, and is adaptable for field-sustainable formulation. Assay test results will be submitted to the Armed Forces Pest Management Board (AFPMB), Silver Spring, Md. for approval as the Department of Defense (DoD) methodology for dengue virus vector surveillance on the RAPID-based Vector Surveillance Analytic System (VSAS) and to the Joint Projects Office, DoD for approval as a candidate assay for FDA clearance on DoD approved instrumentation, the Joint Biological Agent Identification and Diagnostic System (JBAIDS) [Idaho Technology, Inc., Salt Lake City, Utah]. The example of the McAvin assay described here was initially developed in a wet reagent format, but also has been successfully placed in a freeze-dried, room temperature stable, hydrolytic enzyme resistant, format. The example of the McAvin assay described here utilizes dual fluorogenic probe (TaqMan) hydrolysis reverse transcriptase-polymerase chain reaction (RT-PCR).

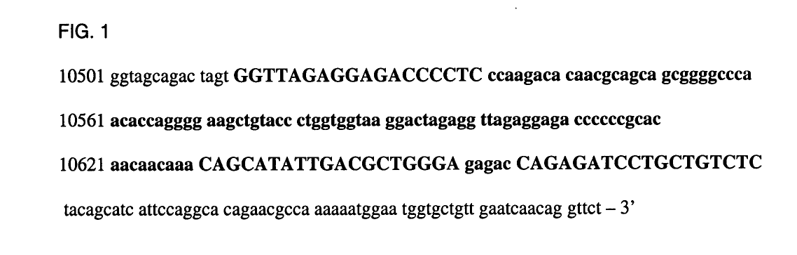

FIG. 1 is a diagram showing examples of a target sequence (in bold type), RT-PCR forward primer (sense), probe (anti-sense), and reverse primer (anti-sense) sequences (in bold caps). The target sequence is at the extreme 3′ untranslated region (UTR) of the dengue virus genome, GenBank accession number U88536 at base count 10515-10671 (in bold type). Target sequence: GGTTAGAGGAGACCCCTC ccaagaca caacgcagca gcggggccca acaccagggg aagctgtacc ctggtggtaa ggactagagg ttagaggaga ccccccgcac aacaacaaa CAGCATATTGACGCTGGGA gagac CAGAGATCCTGCTGTCTC (SEQ ID NO:1).

In FIG. 1, the RT-PCR forward primer (sense), probe (anti-sense), and reverse primer (anti-sense) sequences are shown in bold caps, respectively. Primer and probe sequences are conserved across dengue virus 1-4 serotypes and exclude genotypically and clinical significant organisms (McAvin et al. 2005).

Assay primer and probe sequences were selected by aligning homologous genomic regions of serotypes 1-4 that excluded other clinically significant flaviviruses. Alignments were compared visually using the Clustal algorithm (Thompson et al Nucleic Acids Res 1994; November; 11(22): 4673-80) in the MegAlign program of DNA Star software (Perkin Elmer, Norwalk, Conn.) [Clewley et al Methods Mol Biol 1997; 70: 119-29). Maximally conserved sequences were chosen from dengue virus type 1-4 genomes downloaded from Genebank accession numbers U88536, M19197, M93130, AF326825, respectively. The genomic target was defined at the 10553-10717 base sequence of the 3′ non-coding region. Yellow fever, JE, WN, and SLE virus type strain genomic sequences were aligned and visually evaluated to validate heterology with primer and probe sequences, Genebank accession numbers X03700/K02749, M18370, M12294/M10103, AF242895, respectively. The resulting primer and probe oligonucleotide sequences follow:

| | |
|---|---|
| Forward Primer (DUJCM-F1) | |
| 5′-GGT TAG AGG AGA CCC CTC-3′. | (SEQ ID NO:2) |
| Reverse Primer (DUJCM-R1) | |
| 5′-GAG ACA GCA GGA TCT CTG-3′. | (SEQ ID NO:3) |
| Probe (DU-JCM-TM1) | |
| 5′TCC CAG CGT CAA TAT GCT G 3′. | (SEQ ID NO:4) |

“Oligonucleotide” means a polymer of nucleic acids (typically less than 50 nucleotides); the term may include synthetic analogs.

Probe and primers sequence heterology with genomic sequences of closely related species through diverse genera were validated by BLAST database search (BLAST, Madison, Wis.) [Altschul et al J Mol Biol 1990; Oct. 5; 215(3): 403-10]. Melting temperatures were quantified and the absence of significant primer dimerizations and secondary structure (hairpin) formations were confirmed with PrimerExpress software (PE Applied Biosystems, Foster City, Calif.). Primers and probes were synthesized and quality control conducted commercially (Synthetic Genetics, San Diego, Calif.).

Assays were optimized on the Ruggedized Advanced Pathogen Identification Device (R.A.P.I.D.) with a proprietary buffer system (Idaho Technology Incorporated, Salt Lake City, Utah). Wet and field-formatted (lyophilized) assay sensitivity and specificity test methods and results are described in McAvin J C, Escamilla E M, Blow J A, Turell M J, Quintana M, Bowles D E, Swaby J A, Barnes W J, Huff W B, Lohman K L, Atchley D H, Hickman J R, Niemeyer D M: Rapid Identification of Dengue Virus by RT-PCR Using Field-Deployable Instrumentation, Mil Med; 2005 December; 170(12): 1053-9 (hereby incorporated by reference); and in McAvin J C, Blow J A, John L. Putnam J L, Swaby J A: Deployable, Field-Sustainable RT-PCR Assays for Rapid Screening and Serotype Identification of Dengue Virus in Mosquitoes (Mil Med; in press) (hereby incorporated by reference). Master mix components are 0.20 mM concentrations of dATP, dTTP, dGTP, and dCTP (Idaho Technology Incorporated), 2.5 mM Mn(OAc) (Roche Molecular Biochemicals, Indianapolis, Ind.), 2.5 U Tth polymerase (Roche Molecular Biochemicals), and 20% volume to volume of proprietary 5×RT buffer (Idaho Technology Incorporated). Forward primer concentration is 0.50 μM, reverse primer 0.50 μM, and TaqMan probe 0.50 μM. The assay probe (FAM 5′ TCC CAG CGT CAA TAT GCT G 3′ TAMRA) is dual fluorogenic labeled with a 5′ reporter dye, 6-carboxyfluorescein, and 3′ quencher dye, 6-carboxytetramethylrhodamine (Wittwer et al Bio Techniques 1997; January; 22:130-38). A standardized RT-PCR thermal cycling protocol was established that consisted of RT at 60° C. for 20 minutes followed by an initial cDNA denaturation at 94° C. for 2 minutes, and PCR for 45 cycles at 94° C. for 0 seconds of template denaturation and 60° C. for 20 seconds of combined annealing and primer extension.

The example of the McAvin assay described here is a singleplex reaction vs. multiplex reactions of U.S. Pat. No. 6,855,521 to Callahan and U.S. Pat. No. 6,793,488 to Houng. The McAvin assay was designed using three non-degenerate oligonucleotides; a single forward primer, a single reverse primer, and a single probe. Callahan is comprised of four fundamental oligonucleotides with sequence degeneracy in the forward and reverse primers that requires an additional three oligonucleotides for a total of seven. The Houng reaction requires eight oligonucleotides. Multiplexing is an extremely daunting process because each oligonucleotide concentration represents a separate variable that must be considered in the assay optimization process, each oligonucleotide has the potential to interfere with the other oligonucleotides during the PCR, and analytic instrumentation capable of detecting fluorescence of multiple dyes must be used. Multiplexing a freeze-dried formulation is much more difficult to accomplish than in a wet formulation. While Callahan has shown that his assay has been successfully multiplexed in the wet format it is not known if multiplexing was been attempted in a freeze-dried formulation. Houng has not reported that his reaction has been successfully multiplexed in a wet or freeze-dried formulation.

Wet RT-PCR reagents must be constantly maintained at minus 20° Celsius or degradation occurs within hours therefore require established laboratory and logistic infrastructures. Freeze-dried RT-PCR reagents can be stored at room temperature thus transported and stored without the need of a minus 20° Celsius freezer. The McAvin assay was initially developed in a wet reagent format but has been successfully placed in a freeze-dried, room temperature stable, hydrolytic enzyme resistant, format with a proprietary formulation (Idaho Technology Incorporated, Salt Lake City, Utah). The freeze-dried assay is optimized for use on DOD approved field-deployable instrumentation, the ‘Ruggedized’ Advanced Pathogen Identification Device (RAPID) (Idaho Technology Incorporated, Salt Lake City, Utah). Callahan and Houng multiplex assays have not been reported in a freeze-dried format and both of these assays require laboratory-based instrumentation.

Callahan expressed skepticism about a singleplex assay: “The design of the dengue group assay required a different approach due to the lack of a sequence homology among the four serotypes of sufficient length to serve as an assay target . . . a multiplex format . . . was used.” See U.S. Pat. No. 6,855,521 to Callahan, Column 6, starting at Line 15.

McAvin Assay Sensitivity and Specificity Test Results

Assay Sensitivity and Specificity Testing with Flavivirus Reference Strains

Preliminary assay sensitivity and specificity evaluations were conducted with a known panel of total nucleic acid extracts from dengue serotypes 1-4 infected Aedes aegypti inoculated with three different strains of dengue 1, eight strains of dengue 2, three strains of dengue 3, three strains of dengue 4, and a cross-reactivity test panel consisting of total nucleic acid extracts from multiple strains of other Flaviviridae (three strains each of YF, JE, WN, and four strains of SLE). In vitro sensitivity and specificity of the assay was 100% concordant: DU-JCM ( 17/17) and ( 13/13), respectively. No cross-reactivity was observed with vector species.

Assay Sensitivity and Specificity Testing with Mosquito Panels

Testing of assay in vitro sensitivity and specificity were accomplished with a blind panel of; 27 dengue-infected mosquitoes (six dengue 1 infected mosquitoes, five dengue 2, ten dengue 3, six dengue 4), 21 non-dengue virus (seven YF virus-infected Aedes aegypti, and seven each of WN and SLE virus-infected Culex spp) infected mosquitoes, and 11 uninfected mosquitoes or 27 positives and 32 negatives (Table 1). Diluent samples were not included in statistical analysis. Assay results were; DU-JCM sensitivity 100% ( 27/27) and specificity 94% ( 30/32). The DU-JCM assay reported two false positives—panel ID numbers 110 and 114 (Table 1). That both DU-JCM and a DEN-1 specific RT-PCR assay reported panel ID number 114 as dengue virus positive implies experimental error. Additional testing will be done to further delineate observed results. No cross-reaction was observed with vector species genomic DNA and medium diluent. Sample processing and RT-PCR required less than two hours.

Assay Sensitivity and Specificity Testing with Clinical Specimens

Testing was accomplished with a blind panel of eight dengue viremic (dengue 2) and 31 non-dengue infected febrile patient sera specimens (Table 2). Dengue virus universal assay (DU-JCM) in vitro sensitivity was 100% ( 8/8) and specificity 100% ( 31/31) when tested against the human sera panel. Human genomic DNA displayed no detectable fluorescence above background. Sample processing and real-time RT-PCR required less than two hours.

TABLE 1
RT-PCR Results of Dengue Virus Assay
Testing of Infected Mosquitoes

| Panel | | | | |
|---|---|---|---|---|
| ID | Virus | Strains | pfu/leg | DU-JCM |
| 101 | Dengue-1 | Hawaii | 3 | Positive |
| 104 | Dengue-4 | H241 | 3.6 | Positive |
| 105 | none | | 1 Uninfected | — |
| 106 | Dengue-1 | Hawaii | 3.2 | Positive |
| 109 | Dengue-4 | H241 | 3.6 | Positive |
| 110 | none | | 1 Uninfected | Positive |
| 112 | Dengue-4 | H241 | 3 | Positive |
| 113 | Dengue-1 | Hawaii | 2.9 | Positive |
| 114 | none | | 1 Uninfected | Positive |
| 117 | Dengue-4 | H241 | 2.6 | Positive |
| 118 | none | | 1 Uninfected | — |
| 120 | Dengue-1 | Hawaii | 3 | Positive |
| 121 | none | | 1 Uninfected | — |
| 123 | Dengue-4 | H241 | 3.3 | Positive |
| 124 | Dengue-1 | Hawaii | 3 | Positive |
| 125 | none | | 1 Uninfected | — |
| 127 | Dengue-4 | H241 | 3.1 | Positive |
| 128 | Dengue-1 | Hawaii | 3 | Positive |
| 401 | Dengue-3A | H87 | 2 | Positive |
| 402 | Dengue-2 | S16803 | 3.9 | Positive |
| 403 | Dengue-3B | CH53489 | 3.6 | Positive |
| 404 | none | | 1 Uninfected | — |
| 405 | Dengue-2 | S16803 | 4.2 | Positive |
| 406 | Dengue-3A | H87 | 3.6 | Positive |
| 407 | Dengue-2 | S16803 | 3.6 | Positive |
| 408 | Dengue-3B | CH53489 | 4 | Positive |
| 409 | none | | 1 Uninfected | — |
| 410 | none | | 1 Uninfected | — |
| 411 | Dengue-3A | H87 | 2.8 | Positive |
| 412 | Dengue-3B | CH53489 | 4 | Positive |
| 413 | Dengue-2 | S16803 | 4.3 | Positive |
| 414 | none | | 1 Uninfected | — |
| 415 | Dengue-2 | S16803 | 4.3 | Positive |
| 416 | Dengue-3B | CH53489 | 3.9 | Positive |
| 417 | Dengue-3A | H87 | 2.2 | Positive |
| 418 | none | | 1 Uninfected | — |
| 419 | Dengue-3B | CH53489 | 3 | Positive |
| 420 | Dengue-3A | H87 | 3 | Positive |
| 201 | Yellow fever | Asibe | 4 | — |
| 202 | St. Louis encephalitis | Ft. Washington | 5 | — |
| 203 | West Nile | Crow 397-99 | >5 | — |
| 204 | Diluant | | 0 | — |
| 205 | Yellow fever | Asibe | 3.9 | — |
| 206 | St Louis encephalitis | Ft Washington | 5 | — |
| 207 | West Nile | Crow 397-99 | >5 | — |
| 208 | Diluant | | 0 | — |
| 209 | West Nile | Crow 397-99 | >4.5 | — |
| 210 | Diluant | | 0 | — |
| 211 | Yellow fever | Asibe | 3.7 | — |
| 212 | St. Louis encephalitis | Ft. Washington | >4.0 | — |
| 213 | West Nile | Crow 397-99 | >4.5 | — |
| 214 | St. Louis encephalitis | Ft. Washington | >4.0 | — |
| 215 | West Nile | Crow 397-99 | >4.5 | — |
| 216 | Diluant | | 0 | — |
| 217 | Yellow fever | Asibe | 4 | — |
| 218 | St. Louis encephalitis | Ft. Washington | >4.0 | — |
| 219 | Yellow fever | Asibe | 3.1 | — |
| 220 | Diluant | | 0 | — |
| 221 | West Nile | Crow 397-99 | >4.5 | — |
| 222 | St. Louis encephalitis | Ft. Washington | >4.0 | — |
| 223 | Yellow fever | Asibe | 4 | — |
| 224 | Diluant | | 0 | — |
| 225 | West Nile | Crow 397-99 | >4.5 | — |
| 226 | Yellow fever | Asibe | 4 | — |
| 227 | St. Louis encephalitis | Ft. Washington | >4.0 | — |
| 228 | Diluant | | 0 | — |

TABLE 2
RT-PCR Results of Dengue Virus Assay Testing of Human Serum

| No. | Sample ID | Collection Date | Serology | DU-JCM |
|---|---|---|---|---|
| 1 | 02-2935. | 7 Jul. 2002 | — | — |
| 2 | 02-2938. | 7 Jul. 2002 | — | — |
| 3 | 02-2945. | 5 Jul. 2002 | — | — |
| 4 | 02-2948. | 7 Jul. 2002 | — | — |
| 5 | 02-2943. | 6 Jul. 2002 | — | — |
| 6 | 02-2964. | 3 Jul. 2002 | — | — |
| 7 | 02-2965. | 4 Jul. 2002 | — | — |
| 8 | 02-2958. | 7 Jul. 2002 | — | — |
| 9 | 02-2963. | 3 Jul. 2002 | Dengue 2 | Positive |
| 10 | 02-2968. | 3 Jul. 2002 | — | — |
| 11 | 02-2934. | 6 Jul. 2002 | — | — |
| 12 | 02-2955. | 7 Jul. 2002 | Dengue 2 | Positive |
| 13 | 02-2957. | 6 Jul. 2002 | Dengue 2 | Positive |
| 14 | 02-2960. | 3 Jul. 2002 | — | — |
| 15 | 02-2932. | 6 Jul. 2002 | — | — |
| 16 | 02-2931. | 7 Jul. 2002 | — | — |
| 17 | 02-2933. | 5 Jul. 2002 | — | — |
| 18 | 02-2942. | 7 Jul. 2002 | — | — |
| 19 | 02-2921. | 5 Jul. 2002 | — | — |
| 20 | 02-2888. | 3 Jul. 2002 | — | — |
| 21 | 02-2882. | 5 Jul. 2002 | Dengue 2 | Positive |
| 22 | 02-2878. | 4 Jul. 2002 | — | — |
| 23 | 02-2972. | 3 Jul. 2002 | — | — |
| 24 | 02-2909. | 2 Jul. 2002 | — | — |
| 25 | 02-2978. | 08 Jul. 2002 | — | — |
| 26 | 02-2117. | 27 Jun. 2002 | — | — |
| 27 | 02-2115. | 27 Jun. 2002 | Dengue 2 | Positive |
| 28 | 02-1720. | 23 Jun. 2002 | — | — |
| 29 | 02-2238. | 1 Jul. 2002 | Dengue 2 | Positive |
| 30 | 02-2969. | 1 Jul. 2002 | — | — |
| 31 | 02-2144. | 30 Jun. 2002 | Dengue 2 | Positive |
| 32 | 02-2114. | 29 Jun. 2002 | — | — |
| 33 | 02-2101. | 26 Jun. 2002 | Dengue 2 | Positive |
| 34 | 02-2125. | 1 Jul. 2002 | — | — |
| 35 | 02-1846. | 25 Jun. 2002 | — | — |
| 36 | 02-2874. | 4 Jul. 2002 | — | — |
| 37 | 02-2116. | 27 Jun. 2002 | — | — |
| 38 | 02-2877. | 4 Jul. 2002 | — | — |
| 39 | 02-2869. | 4 Jul. 2002 | — | — |

Note:
All patient samples were collected during the acute febrile phase.

### Further Working Examples

The example of the McAvin assay described here has been used in vector surveillance by the Environmental Science Division, United States Army Center for Health Promotion and Preventative Medicine (USACHPPM), Fort Lewis, Wash. 98433-9500 and Entomology Science Division, USACHPPM, Fort George G. Meade, Md. 20755-5225. Follow-on validation testing was successfully completed in August 2007, through a collaborative effort including the Air Force Institute for Operational Health and Walter Reed Army Institute of Research (WRAIR), Silver Spring, Md. and the Department of Entomology, Armed Forces Research Institute of Medical Sciences (AFRIMS), Bangkok, Thailand. Laboratory and field validation testing were conducted with relevant specimens. In laboratory testing, assay limit of detection was established at >7 to ≦70 genomic equivalents (McAvin et al 2007). Assay sensitivity was 100% ( 16/16) and specificity was 100% ( 20/20) for a blind panel (n=36) of three strains each of dengue virus 1 through 4 isolates (n=12), dengue virus 1-4 infected Ae. aegypti (n=4), non-infected Ae. Aegypti (n=1), Plasmodium vivax (n=5), P. falciparum (n=5), P. v. and P.f. mixed (n=5) infected and non-infected An. dirus (n=4). In field testing, two dengue infected mosquitoes were detected in a panel (n=28) of female Ae. Aegypti collected near homes of DF/DHF diagnosed patients. One specimen was determined positive (Ct=19) by the assay on the RAPID and confirmed by gold standard methodology, indirect immunofluorescence (IFA) and gel electrophoresis. Results were reproduced by traditional PCR. The second specimen was positive (Ct=32) by the assay on the RAPID and confirmed by gel electrophoresis. Testing by IFA and traditional PCR were negative. Assay performance under austere field conditions was confirmed using a control panel (n=12) of three strains each dengue virus 1-4 isolates (n=12). A truck battery and portable generator were used as power sources for the VSAS. Sample processing and RT-PCR required less than two hours. These data clearly demonstrate that the assay when used on the VSAS provides an efficacious method for real-time, deployable vector surveillance and suggest that assay sensitivity may exceed IFA. Follow-on sensitivity testing comparing the assay to IFA is planned.

In collaboration with the Viral Diseases Department, Naval Medical Research Center, Silver Spring, Md. and Department of Virology, AFRIMS laboratory testing was conducted with archived clinical specimens. The assay was tested with 102 dengue patient serum samples from Peru and Indonesia confirmed by clinical symptoms and gold-standard methodology, virus isolation. Sensitivity was 97% ( 99/102). Specificity was 98% ( 49/50) with dengue virus negative serum samples. For patient samples from Thailand, assay sensitivity was 100% ( 32/32) and specificity was 100% ( 8/8) in testing with a blind panel (n=40) of dengue patient serum spiked-cultures (n=32), Japanese encephalitis patient serum spiked-cultures (n=5), and culture diluent (n=3). No cross-reactivity occurred in testing with a panel (n=10) of scrub typhus patient serum samples. The McAvin assay has been used within the Department of Defense by Department of Defense employees, not for commercial purposes. For example, the assay has been used by Dr. Alexandra Spring, a civilian employee of the Army, at Fort Meade, Md., and by Dr. Miguel Quintana, an Army reservist, at Fort Lewis, Wash. and in Central America. Dr. Quintana was helped by local technicians in Central America who collected samples, but they did not use the assay to analyze the samples.

In summary, the examples provided here address a need for simpler Dengue assay technology, adaptable to field-deployable Dengue virus surveillance and DF/DHF diagnoses, for example.

The examples provided herein are intended to demonstrate only some embodiments of the invention. Other embodiments may be utilized and structural changes may be made, without departing from the present invention.

I claim:

1. A method of hybridization, said method comprising
providing a single-stranded oligonucleotide at least 26 nucleotides in length, the oligonucleotide being complementary to a portion of SEQ ID NO:1; and
contacting the oligonucleotide with a nucleic acid comprising the sequence of SEQ ID NO:1, under conditions that permit hybridization of the oligonucleotide with the nucleic acid.

2. A method of hybridization, said method comprising
providing a single-stranded oligonucleotide comprising the sequence of SEQ ID NO:4; and
contacting the oligonucleotide with a nucleic acid comprising the sequence of SEQ ID NO:1, under conditions that permit hybridization of the oligonucleotide with the nucleic acid.

3. The method of claim 2, further comprising
employing PCR primer (DUJCM-F1) 5′-GGT TAG AGG AGA CCC CTC-3′ (SEQ ID NO:2).

4. The method of claim 2, further comprising
employing PCR primer (DUJCM-R1) 5′-GAG ACA GCA GGA TCT CTG-3′ (SEQ ID NO:3).
